(19) United States
(12) Patent Application Publication
(10) Pub. No.: US 2007/0082035 A1
(43) Pub. Date: Apr. 12, 2007

(54) Anti-infective hygiene products based on cellulose acetate phthalate

(75) Inventors: Alexander Robert Neurath, New York, NY (US); Nathan Strick, Oceanside, NY (US); Yun-Yao Li, Fresh Meadows, NY (US)

Correspondence Address: FRISHAUF, HOLTZ, GOODMAN & CHICK, PC, 220 Fifth Avenue, 16TH Floor, NEW YORK, NY, 10001-7708, US

(73) Assignee: NEW YORK BLOOD CENTER, INC., New York, NY (US)

(21) Appl. No.: 11/542,085

(22) Filed: Oct. 3, 2006

Related U.S. Application Data
(60) Provisional application No. 60/724,625, filed on Oct. 6, 2005.

(51) Int. Cl. A61K 31/717; A61F 13/20

(52) U.S. Cl. 424/443; 514/57; 604/358

(57) ABSTRACT

Methods for maintaining a low vaginal pH, preventing the propagation of toxic shock syndrome toxin 1 production during menstruation, preventing the shedding and dissemination of a herpesvirus and treating herpes lesions comprising administering to a human a formulation comprising a pharmaceutically effective amount of an insoluble micronized form of cellulose acetate phthalate or a cellulose acetate phthalate film or a cellulose acetate phthalate porous sponge. Also a method of treating Candida albicans infection comprising administering to a human female a pharmaceutically effective amount of a composition comprising micronized cellulose acetate phthalate and miconazole nitrate.

### CROSS-REFERENCE TO RELATED APPLICATION

This application claims the benefit of priority under 35 USC 119(e) for provisional application Ser. No. 60/724,625 filed Oct. 6, 2005, the entire contents of which are incorporated by reference herein.

### GOVERNMENT RIGHTS

This invention was made with United States government support under Grant 1U19HD048957 from the National Institute of Health. The United States government has certain rights in this invention.

### BACKGROUND OF THE INVENTION

1. Field of the Invention

The present invention concerns an insoluble micronized form of cellulose acetate phthalate (“CAP”) (such as in the form of a gel) or a solid dosage form of CAP which is dispersible in water and generates micronized CAP particles (such as a film or sponge) as an active ingredient in anti-infective/hygiene products intended for long-term repeated use.

The present invention further relates to the combined use of cellulose acetate phthalate and Lactobacilli to maintain a normal acidic vaginal environment.

The present invention is also directed to preventing toxic shock syndrome toxin 1 production during menstruation.

The present invention also serves to prevent the shedding and dissemination of a herpesvirus, particularly herpesvirus type 2, and to treat herpes lesions.

The present invention is further directed to maintaining a low vaginal pH.

The present invention also concerns a method to treat Candida albicans infections.

2. Background Information

a. Cellulose Acetate Phthalate Cellulose acetate 1,2-benzenedicarboxylate (Cellacephate, cellulose acetate phthalate, Cellulosi acetas phthalas, Cellacefate; CAP) which has been widely used for enteric coating of tablets and capsules in the pharmaceutical industry (Goskonda S R, Lee J C, “Cellulose acetate phthalate,” in Handbook of Pharmaceutical Excipients, Ed. A. H. Kibbe. Third Edition, American Pharmaceutical Association/Pharmaceutical Press, Washington, D.C./London,U.K., pp. 99-101, (2000)), is also an active ingredient in distinct formulations of candidate microbicides expected to prevent the transmission of several sexually transmitted disease (STD) pathogens including the human immunodeficiency virus (HIV-1), herpesvirus and several bacterial STD pathogens (Neurath A R, Debnath A K, Jiang S, Strick N, Dow G J, “Method for decreasing the frequency of transmission of viral infections using cellulose acetate phthalate or hydroxypropyl methylcellulose phthalate excipients”, U.S. Pat. No. 5,985,313; Neurath A R, Jiang S, Debnath A K, Strick N, Dow G J, “Methods and compositions for decreasing the frequency of HIV, herpesvirus and sexually transmitted bacterial infections”, U.S. Pat. No. 6,165,493; Neurath AR, “Method for inactivating bacteria associated with bacterial vaginosis using cellulose acetate phthalate and/or hydroxypropyl methylcellulose phthalate”, U.S. Pat. No. 6,462,030; Neurath AR, Strick N, “Biodegradable microbicidal vaginal barrier device”, U.S. Pat. No. 6,572,875; Neurath AR, Strick N, “Biodegradable microbicidal vaginal barrier device”, U.S. Pat. No. 6,596,297; Neurath A R, Strick N, Li Y Y, “Water dispersible film,” US Pat. Application Publication No. 2005/0070501 A1 (published March 21, 2005)).

b. Effects of Lactobacilli

Appropriate strains of Lactobacilli colonizing human mucosal sites have a beneficial health effect and inhibit the colonization by undesirable pathogenic bacteria including bacterial STD pathogens (Hughes V L, Hillier S L, “Microbiologic characteristics of Lactobacillus products used for colonization of the vagina,” Obstetrics & Gynecology, 75:244-48 (1990); (Reid G, “Microbial adhesion to biomaterials and infections of the urogenital tract,” Colloids and Surfaces B: Biointerfaces, 2:377-385 (1994); Reid G, “The scientific basis for probiotic strains of Lactobacillus,” Appl. Environ, Microbiol. 6:3763-3766 (1999); Reid G, Bruce A W, Bussher H J, Van der Mei H C, “Lactobacillus therapies,” U.S. Pat. No. 6,051,552; Bruce A W, Reid G, “Lactobacillus compositions and methods for treating urinary tract infections,” U.S. Pat. No. 6,180,100 ; Cavaliere Ved. Vesely R M A, De Simone C, “Pharmaceutical compositions containing lactobacilli for treatment of vaginal infections and related method,” U.S. Pat. No. 6,277,370; Reid G, Bruce A W, Oral adinistration of lactobacillus for the treatment and prevention of urogenital infection,” US Pat. Application No. 2002/0044926 (published Apr. 18, 2002); Bruce A W, Reid G, “Oral administration of lactobacillus for the treatment and prevention of urogenital infection,” U.S. Pat. No. 6,479,051; Mercenier A, Pavan S, Pot B, “Probiotics as biotherapeutic agents: Present knowledge and future prospects,” Curr. Pharm. Des., 9:99-110 (2002); Chrisope G L, “Vaginal lactobacillus medicant,” U.S. Pat. No. 6,468,526; Reid G, Bruce A W, “Oral administration of lactobacillus for the maintenance of health in women during pregnancy and at other life stages, to reduce the risk of urogenital diseases,” US Pat. Application No. 2003/0118571 (published Jun. 26, 2003); Hakansson E G, Forsgren-Brusk U, Anderson R, Holm S E, Hakansson S, “Strain of Lactobacillus plantarum and uses thereof,” U.S. Pat. No. 6,761,885).

It has been previously established that formulations of CAP inactivate several sexually transmitted bacteria but have no effect on Lactobacillus crispatus within a time period of 5 or 15 minutes (Neurath A R, Jiang S, Debnath A K, Strick N, Dow G J, “Methods and compositions for decreasing the frequency of HIV, herpesvirus and sexually transmitted bacterial infections”, U.S. Pat. No. 6,165,493). The results of these short-term inactivation experiments have not established whether Lactobacilli differed from bacteria associated with STDs and bacterial vaginosis (Neurath A R, “Method for inactivating bacteria associated with bacterial vaginosis using cellulose acetate phthalate and/or hydroxypropyl methylcellulose phthalate”, U.S. Pat. No. 6,462,030), respectively, only with respect to inactivation kinetics, or whether Lactobacilli maintain long-term viability in the presence of micronized cellulose acetate phthalate.

c. Toxic Shock Syndrome Toxin I (TSST-1) Production

The vaginal environment of reproductive age women is acidic (pH 3-5). In the course of menstruation, the vaginal environment acquires an increased pH due to the presence of menstrual fluid and blood. This can result in changes in the composition of microorganisms residing in the vaginal environment and in a decreased level of Lactobacilli and the increased probability of colonization by pathogenic microorganisms (Brzezinski A, Stem T, Arbel R, Rahav G, Benita S, “Efficacy of a novel pH-buffering tampon in preserving the acidic vaginal pH during menstruation,” Intemat. J. Gynecology, 85:298-300 (2004)). One of the possible consequences is associated with infection and colonization by Staphylococcus aureus bacteria and the associated production of TSST-1. To address this problem, absorbent products such as tampons, sanitary napkins, wound dressings, etc., containing ingredients which could suppress the replication of S. aureus have been designed and developed, e.g., tampons containing esters of polyhydric aliphatic alcohols and fatty acids have been developed (Brown-Skrobot S K, “Additives to tampons”, U.S. Pat. No. 5,705,182). Information regarding the association of TSST-1 with the use of tampons and the discovery of additives to tampons expected to prevent TSST-1 formation is summarized in the text of the aforementioned US patent and included herein.

Subsequent to the publication of reports associating toxic shock syndrome with the use of tampons, a number of investigators undertook studies designed to evaluate the effect of tampons on growth of S. aureus bacteria, as well as the effect of tampons on the production of TSST-1 by those bacteria. Early efforts to elucidate the role of tampons in TSS yielded conflicting data. Schlievert et al. (Schlievert P M, Blomster D A, Kelly J A, “Toxic shock syndrome Staphylococcus aureus: Effect of tampons on toxic shock syndrome toxin 1 production,” Obstet. Gynecol., 64:666-671 (1984)) studied the effect of tampons on S. aureus to evaluate whether or not tampon components increase growth of S. aureus and production of toxic shock syndrome toxin-1. It was concluded that, under the test conditions of their study, tampon components provide neither nutrients for growth of toxic shock syndrome S. aureus nor factors that induce production of toxic shock syndrome toxin-I above control levels. After six hour incubation, some commercially available tampons which were tested were inhibitory to bacterial growth and suppressed toxin production. Others suppressed toxin production but did not inhibit cell growth. One tampon inhibited cell growth but increased the amount of toxin produced. On the other hand, Tierno P M Jr, Hanna B A, “In vitro amplification of toxic shock syndrome toxin-1 by intravaginal devices,” Contraception, 31:185-194 (1985) reported that in their experiments tampons did stimulate S. aureus to produce TSST-1.

Reiser et al. (Reiser R F, Hinzman S J, Bergdoll M S, “Production of toxic shock syndrome toxin 1 by Staphylococcus aureus restricted to endogenous air in tampons,” J. Clin. Microbiol., 25:1450-1452 (1987)) thereafter reported the results of tests they conducted to determine the effect of four brands of tampons on production of toxic shock syndrome toxin-1. The amount of air available to the tampons which were tested was limited to that contained in sacs (made from cellulose sausage casing with a molecular weight cut-off of less than 10,000) in which the tampons were enclosed during testing. This method was deemed advantageous in that the limited amount of available air was thought to mimic more closely than previously used methods the in vivo condition in the vagina during menstruation with a tampon in place and in that the tampons which were tested were not altered prior to testing. The results of the tests conducted by Reiser et al. indicated that tampons provide increased surface area for the S. aureus bacteria to grow and adequate oxygen for toxin production. No significant inhibition of growth of the staphylococci bacteria or TSST-1 production by any of the tampons tested was noted.

Robbins et al. (Robbins R N, Reiser R F, Hehl G L, Bergdoll M S, “Production of toxic shock syndrome toxin 1 by Staphylococcus aureus as determined by tampon disk-membrane-agar method,” J. Clin. Microbiol., 25:1446-1449 (1987)) at the same time as Reiser et al., reported the effect of 17 commercially available tampons on TSST-1 toxin production using a disk-membrane-agar (DMA) method, with incubation at 37° C. for 19 hours under 5 vol. % CO₂ in air. Filter membranes overlaying agar medium (with or without blood) in small petri dishes were inoculated with a TSST-1 producing strain of S. aureus. Robbins et al. concluded that the main role of tampons in TSS may be that of providing a fibrous surface for heavy colonization and sufficient air for TSST-1 production. In addition, they found evidence of inhibition of TSST-1 production by additives such as the deodorant/surfactant used in a commercially available deodorant tampon and a decrease in TSST-1 production by inhibiting growth of S. aureus was observed in the case of a different commercially available tampon. It was thought that both inhibition of TSST-1 production and inhibition of S. aureus growth might prove to be important in reducing the risk of TSS.

U.S. Pat. No. 4,405,323 to Auerbach, “Sanitary napkin,” the entire contents of which are hereby incorporated by reference herein, discloses a tampon designed to eliminate the hazards of toxic shock syndrome and dysmenorrhea. The tampon has incorporated therein an antibacterial agent which is said to disperse on contact with body fluids and prevent development of the organisms which produce the toxins which cause toxic shock syndrome. Among the antibacterial materials disclosed for use are povidone-iodine compound, mercury, zinc, penicillin, erythromycin and nitrofurazone.

WO 86/05388 (published Sep. 25, 1986) to Kass teaches that the inclusion of a salt of a nontoxic divalent cation in absorptive pads, e.g., catamenial tampons, inhibits production of toxic shock syndrome toxin-1 and other staphylococcal products during use of said absorptive pad. Suitable salts include those of magnesium, barium, calcium or strontium (preferred) or of other divalent cations such as zinc, manganese, copper, iron, nickel and the like. The anionic portion of the salt is not critical. Magnesium stearate and magnesium acetate are particularly preferred salts for use in the invention.

In U.S. Pat. No. 4,374,522 to Olevsky, “Tampon with central reservoir,” the entire contents of which are hereby incorporated by reference herein, it is stated that patterns of use of catamenial tampon seem to indicate that high absorptive capacity with the concomitant extended period of use of certain tampons are factors which contribute to the formation of toxic shock syndrome. The Olevsky patent theorizes that tampons having limited absorptive capacity and requiring relatively more frequent changes may be desirable. The Olevsky patent provides a tampon made of conventional cellulosic materials, such as rayon fibers, which are compressed into a bullet-shape with an open bottom surface sealed by a fluid impermeable sheet. The fluid impermeable bottom and the traditional bullet shaped pledget define a hollow core central reservoir area which is said to serve as a reservoir for excess menstrual fluid.

U.S. Pat. No. 4,431,427 to Lefren et al., “Tampons and their manufacture,” the entire contents of which are hereby incorporated by reference herein, discloses menstrual tampons comprising physiologically safe, water-soluble acids in their monomeric, oligomeric or polymeric forms. Citric, glycolic, malic, tartaric and lactic acids are disclosed as being useful in the practice of the invention. The presence of one or more of the above-noted acids in a tampon is said to inhibit the growth of bacteria responsible for toxic shock. Where an acid is used in its polymeric form, the tampon may additionally include an enzyme to hydrolyze the polymeric acid to its monomeric form.

Canadian Patent No. 1,123,155 to Sipos, “Catamenial tampon,” the entire contents of which are hereby incorporated by reference herein, discloses a catamenial tampon for preventing toxic shock syndrome during menstrual flow. The body of the tampon, which is open at the insertion end and is closed at the withdrawal end, is snugly surrounded in its expanded condition by a fluid proof, thin and flexible membrane. This membrane, which can be made of polyethylene sheet, is biased against the vaginal wall during use of the tampon, is neutral to the vaginal mucosa and is completely impermeable to bacteria, viruses and toxic decomposition products of the menstrual flow.

Canadian Patent No. 1,192,701 to Bardhan, “Tampon having means for combating harmful effects of bacteria,” discloses a tampon for the absorption of menstrual flow and comprising an inner layer of liquid-absorbent material and an outer layer which surrounds and encloses the inner layer. Menstrual discharge may flow inwardly to the inner layer but the outer layer is impervious to the passage of menstrual fluid outwardly from the inner layer. A plurality of liquid absorbent wicks extending from the inner layer through apertures formed in the outer layer serve as conduits for the flow of menstrual discharge from outside the tampon to the inner layer thereof. The disclosed structure is said to minimize the availability of discharge outside the tampon with a resulting reduction in the likelihood of growth of S. aureus and consequently its production of toxin. This patent also discloses that an antimicrobial compound which is bactericidal or bacteriostatic to S. aureus may be included in the inner layer. The antimicrobial agent may take the form of an antibiotic (such as penicillin, erythromycin, tetracycline or neomycin), a chemotherapeutic agent (such as sulfonamide) or a disinfectant (such as phenol). The patent states that since the tampon is protected by its outer layer from contact with the vaginal wall, the risk of an allergic or other adverse reaction to the anti-microbial agent is minimized, and since the antimicrobial agent is also protected by the outer layer from contact with menstrual discharge, there is little risk of the destruction of commensal organisms in the vagina or development of resistance to the antimicrobial agent by S. aureus in any menstrual discharge outside the vagina.

Notermans et al., “Effect of glyceryl monolaurate on toxin production by clostridium botulinum in meat slurry,” J. Food Safety, 3:83-88 (1981) reported that glyceryl monolaurate, when used in the proportion of 5 g per kg. of meat slurry (pH 6.0-6.2) inhibited toxin productions by Clostridium botulinum type A, type B and type E. This article does not mention Staphylococcus aureus nor any toxin(s) produced therefrom nor does it mention absorbent products or toxic shock syndrome.

U.S. Pat. No. 4, 585,792 to Jacob et al., “Protective additive to vaginal products and catamenials,” the entire contents of which are hereby incorporated by reference herein, discloses that L-ascorbic acid when topically applied to the vaginal area of a human female during menses will inactivate toxins known to contribute to Toxic Shock Syndrome. The ascorbic acid compound may be carried by a vaginal tampon. The disclosure of U.S. Pat. No. 4,722,937, the entire contents of which are hereby incorporated by reference herein, is to the same effect.

U.S. Pat. No. 4,413,986 to Jacobs, “Tampon assembly with means for sterile insertion,” the entire contents of which are hereby incorporated by reference herein, discloses a sterilely-packaged tampon assembly for sterile insertion of a tampon into the vagina and having a guide tube telescoped around an insertion tube and a flexible sheath attached to the inner end of the guide tube and tucked into the inner end of the insertion tube. In use, as the insertion tube is pushed through the guide tube and into the vagina, the flexible sheath is pulled over the inner end of the insertion tube and extends along the exterior thereof. The portion of the insertion tube which is inserted into the vagina is at all times fully sheathed by the flexible sheath.

Additional tampon additives expected to inhibit the production of TSST-1 are alkyl polyglycoside (Resheski-Wedepohl K L, Syverson R E, “Compositions for the inhibition of exoprotein production from gram positive bacteria,” U.S. Pat. No. 6,531,435, the entire contents of which are hereby incorporated by reference herein; Resheski-Wedepohl K L, Syverson R E, Potts D C, Young M D, Yahiaoui A, “Absorbent articles for the inhibition of exoprotein production from gram postivie bacteria,” U.S. Pat. No. 6,599,521, the entire contents of which are hereby incorporated by reference herein; Resheski-Wedepohl K L, Syverson R E, “Inhibition of exoprotein production from gram positive bacteria,” U.S. Pat. No. 6,656,913; Resheski-Wedepohl K L, Syverson R E, “Method of producing a tampon capable of inhibiting exoprotein production from gram positive bacteria,” the entire contents of which are hereby incorporated by reference herein, US Pat. Application No. 2004/0053856 A1 (published Mar. 18, 2004)), isoprenoids (Syverson R E, Proctor R A, “Isoprenoid compostions for the inhibition of exoprotein production from gram positive bacteria,” U.S. Pat. No. 6,534,548, thiolactomycin or thiomalonate (Syverson R E, Proctor R A, “Methods for inhibiting the production of TSST-1,” U.S. Pat. No. 6,821,999; Syverson R E, Proctor R A, “Methods of inhibiting the TSST-1 production in gram positive bacteria,” US Pat. Application No. 2005/0113448 A1 (published May 26, 2005); Syverson R E, Proctor R A, “Methods for inhibiting the production of TSST-1,” US Pat. Application No. 2003/0157148 A1 (published Aug. 21, 2003)), terpenes and terpenoids (Syverson R E, Proctor R A, “Inhibition of exoprotein production in absorbent articles using isoprenoids,” US Pat. Application No. 2003/0105439 A1 (published Jun. 5, 2003)), cerulenin, triclosan, or hexachlorophene (Syverson R E, Proctor R A, “Absorbent articles containing additives,” US Pat. Application No. 2003/0157149 A1 (published Aug. 21 2003)), oxygen inhibiting agents (Deresiewicz R L, Kasper D L, “Absorbent article, particularly a tampon having additives that reduce toxic shock syndrome toxin production,” U.S. Pat. No. 6,548,552, the entire contents of which are hereby incorporated by reference herein) and various aromatic compositions (Syverson R E, Proctor R A, “Inhibition of exoprotein production in absorbent articles using aromatic compositions,” US Pat. Application Publication No. 2003/0135173 A1 (published Jul. 17, 2003)).

d. Topical Treatment of HSV Infections

Herpesviruses include the following viruses isolated from humans:
(1) herpes simplex virus (“HSV-1”)
(2) herpes simplex virus (“HSV-2”)
(3) human cytomegalovirus (“HCMV”)
(4) varicella-zoster virus (“VZV”)
(5) Epstein-Barr virus (“EBV”)
(6) human herpesvirus 6 (“HHV6”)
(7) herpes simplex virus 7 (“HSV-7”)
(8) herpes simplex virus 8 (“HSV-8”).

The lesions caused by herpes simplex virus may appear anywhere on the skin or on mucous membranes, but are most frequent on the face, especially around the mouth or on the lips, conjunctiva and cornea, or the genitals. The appearance of small tense vesicles on an erythematous base follows a short prodromal period of tingling discomfort or itching. Single clusters may vary from 0.5 to 1.5 cm in size, but several groups may coalesce. Herpes simplex on skin tensely attached to underlying structures (for example, the nose, ears or fingers) may be painful. The vesicles may persist for a few days, then begin to dry, forming a thin yellowish crust. Healing usually occurs within 10 days after onset. In moist body areas, healing may be slower, with secondary inflammation. Healing of individual herpetic lesions is usually complete, but recurrent lesions at the same site may result in atrophy and scarring.

In females infected with HSV-2, there may be no skin lesions, the infection may remain entirely within the vagina. The cervix is frequently involved, and there is increasing evidence that this may be a factor in the development of carcinoma of the cervix.

Corneal lesions commonly consist of a recurrent herpetic keratitis, manifest by an irregular dendritic ulcer on the superficial layers. Scarring and subsequent impairment of vision may follow.

Shedding of herpesviruses (HSV) occurs frequently in infected individuals, including those individuals in which the infection is asymptomatic. HSV can be shed from multiple anatomical sites and shedding, similarly to direct exposure to the virus, is a significant risk for HSV transmission (Cherpes T L, Melan M A, Kant J A, Cosentino L A, Meyn L A, Hillier S L, “Genital tract shedding of herpes simplex virus type 2 in women: Effects of hormonal contraception, bacterial vaginosis, and vaginal group B Streptococcus colonization,” Clin. Infect. Dis., 40:1422-1428 (2005); Sacks S L, Griffiths P D, Corey L, Cohen C, Cunningham A, Dusheiko G M, Self S, Spruance S, Stanberry L R, Wald A, Whitley R J, “HSV shedding,” Antiviral Res., 63:S19-S26 (2004); Koelle D M, Wald A, “Herpes simplex virus: The importance of asymptomatic shedding,” J. Antimicrob. Chemother., 45:1-8 (2000)). Since ˜20% of women in the United States, including pregnant women, have been infected with HSV type 2 (HSV-2), there is a high risk of neonatal HSV infection including infection during delivery (Rudnick C M, Hoekzema G S, “Neonatal herpes simplex virus infections,” Am. Fam. Physician, 65:1138-1142 (2002)). HSV shedding and transmission can be suppressed by systemic use of anti-HSV drugs (e.g., acyclovir and valacyclovir) (Scott L L, Hollier L M, McIntire D, Sanchez P J, Jackson G L, Wendel G D Jr, “Acyclovir suppression to prevent clinical recurrences at delivery after first episode genital herpes in pregnancy: An open-label trial,” Infect. Dis. Obstet. Gynecol., 9:75-80 (2002)); Gupta R, Wald A, Krantz E, Selke S, Warren T, Vargas-Cortes M, Miller G, Corey L, “Valacyclovir and acyclovir for suppression of shedding of herpes simplex virus in the genital tract,” J. Infect. Dis., 190:1374-1381 (2004)); Corey L, Ashley R, “Valaciclovir HSV Transmission Study Group,” Herpes, 11:170A-174A (2004); Corey L, Wald A, Patel R, Sacks S L, Tyring S K, Warren T, Douglas J M Jr, Paavonen J, Morrow R A, Beutner K R, Stratchounsky L S, Mertz G, Keene O N, Watson H A, Tai, D, Vargas-Cortes M, “Valacyclovir HSV Transmission Study Group,” N. Engl. J. Med., 35:11-20 (2004)). However, frequent use of these anti-viral drugs might lead to the emergence of drug resistant virus variants (Gershengom H B, Blower S M, “Impact of antivirals and emergence of drug resistance: HSV-2 epidemic control,” AIDS Patient Care STDs, 14:133-142 (2000)). Methods for treatment and prevention of herpetic lesions and associated virus shedding by topical application of some compounds, e.g., vanilloids (O'Neill T P, Kasting G B, Cupps T L, “Use of vanilloids for the prevention of lesions due to herpes simplex infections,” U.S. Pat. No. 5,461,075, tetraethyl ammonium salts and other quaternary ammonium compounds (Docherty J, Tsai C C, “Methods for treating subjects infected with a herpes virus,” U.S. Pat. No. 6,599,945; Johnson B R, “Anti-infective compositions, methods and systems for treating disordered tissue”, U.S. Pat. No. 6,759,434; Baldone J A, “Treatment of diseases caused by herpes viruses”, U.S. Pat. No. 5,158,980), piracetam (Centifanto Y, “Methods of prophylaxis and treatment of herpes simplex lesions utilizing piracetam as the active ingredient,” U.S. Pat. No. 5,232,700), 1 H-imidazole-4-ethanamine, phosphate (IEP) (Jack B A, White B T, “Composition for treatment of cold sores,” U.S. Pat. No. 5,294,440), caffeine (Potts M, “Caffeine in the treatment of herpes simplex virus infections,” U.S. Pat. No. 5,382,436) and zinc salts (Godfrey H R, “Topical zinc compositions and methods of use,” U.S. Pat. No. 6,558,710).

The aforementioned antiviral compounds have the potential to elicit the generation of virus-resistant mutants. All correspond to compounds, the safety of which for repeated human use has not been firmly established. In comparison, the safety of micronized CAP (which has been used in humans for nearly 50 years in the form of tablet and capsule coating material) has already been documented (see above). Because of the exceedingly large surface area per unit weight of micronized CAP, suggesting high adsorptive capacity, it was considered to be important to the present inventors to investigate whether micronized CAP could remove herpesviruses from the environment and thereby block virus shedding. The results of experiments described herein provide supportive evidence for this.

Dual Compartment Applicators

DE 20 2005 004 135 U1(WO 2006/094483) discloses a dual compartment applicator.

U.S. Pat. No. 5,089,606 to Cole discloses a two-component system for vaginal surgery.

U.S. Pat. No. 5,462,526 to Barney discloses squeezing two compartments together to mix the medicaments disposed therein, prior to dispensing the mixture (also see U.S. Pat. No. 6,620,436 to Rolf).

Other U.S. patents which disclose dual compartment dispensers include the following: U.S. Pat. No. 3,760,805 to Higuchi; U.S. Pat. No. 4,180,072 to Sneider; U.S. Pat. No. 4,410,321 to Pearson et al.; U.S. Pat. No. 4,458,733 to Lyons; U.S. Pat. No. 5,531,683 to Kriesel et al.; U.S. Pat. No. 5,569,191 to Meyer; U.S. Pat. No. 5,989,237 to Fowles et al., U.S. Pat. No. 6,090,092 to Fowles et al. and U.S. Pat. No. 6,648,852 to Wirt et al.

### SUMMARY OF THE INVENTION

It is an object of the present invention to provide a tampon to prevent the propagation of Staphylococcus aureus and toxic shock syndrome toxin 1 production during menstruation.

It is also an object of the present invention to maintain a low vaginal pH.

It is another object of the present invention to prevent the shedding and dissemination of herpesvirus, particularly herpesvirus type 2, and to treat herpetic lesions and prevent herpesvirus dissemination.

It is also an object of the present invention to utilize cellulose acetate phthalate formulations in combination with Lactobacilli to maintain a healthy acidic vaginal environment.

Another object of the present invention is to provide for the regular (e.g., daily) application of a cellulose acetate phthalate formulation to maintain a normal acidic vaginal environment and to decrease the replication of harmful microorganisms and viruses in the vagina.

It is a further object of the present invention to employ a dual compartment applicator (dispenser) for providing micronized cellulose acetate phthalate in the form of a water containing gel.

It is another object of the present invention to provide a method for treating Candida albicans infections.

The above objects, along with other objects, aims and advantages, are achieved by the present invention.

The present invention concerns a tampon having insoluble micronized cellulose acetate phthalate (“CAP”) powder being incorporated thereon (or therein) or a tampon combined with a CAP film, the CAP being in a sufficient amount and positioned on (or within) the tampon (such as a tampon wrapped in a CAP film) to prevent the propagation of Staphylococcus aureus and toxic shock syndrome toxin 1 production during menstruation.

The present invention further relates to a method of preventing the propagation of Staphylococcus aureus and toxic shock syndrome toxin 1 production during menstruation comprising vaginally administering to a human female in need thereof a tampon described hereinabove having insoluble micronized cellulose acetate phthalate thereon (or therein) or a cellulose acetate phthalate film wrapper disposed therearound.

The present invention also relates to a method for maintaining a low vaginal pH comprising vaginally administering to a human female a pharmaceutically effective amount of an insoluble micronized form of cellulose acetate phthalate in the form of a gel or a solid dosage form of CAP which is dispersible in water and which generates micronized CAP particles and which is in the form of a CAP film or a CAP sponge.

The present invention further concerns a method for prevention of shedding and dissemination of herpesviruses (particularly herpesvirus type 2 shedded genitally) comprising vaginally administering a pharmaceutically effective amount of an insoluble micronized form of cellulose acetate phthalate in the form of a gel or a solid dosage form of CAP which is dispersible in water and which generates micronized CAP particles and which is in the form of a CAP film.

The present invention is further directed to a method for treating herpes lesions comprising topically administering to a human (female or male) in need thereof a pharmaceutically effective amount of an insoluble micronized form of cellulose acetate phthalate in the form of a gel or topically administering to a human in need thereof a CAP film to a premoistened application site.

The present invention is also directed to a method of maintaining an acidic healthy vaginal environment comprising vaginally administering to a human female pharmaceutically effective amounts of (i) an insoluble micronized form of cellulose acetate phthalate and (ii) Lactobacilli.

The present invention also concerns a method of regularly (e.g., daily) maintaining a normal acidic vaginal environment and decreasing the replication of harmful microorganisms and virus in the vagina comprising administering to a human female a pharmaceutically effective amount of an insoluble micronized form of cellulose acetate phthalate in the form of a gel containing CAP or a CAP film.

An embodiment of the invention is directed to a composition comprising micronized cellulose acetate phthalate and 2 to 5 wt. % of miconazole nitrate.

The present invention is further directed to a method for providing a low vaginal pH, treating herpesvirus, treating a vaginal bacterial infection, blocking toxic shock syndrome generation during menstruation or treating Candida albicans comprising administering to a human female a pharmaceutically effective amount of a micronized form of cellulose acetate phthalate and 2 to 5 wt % of miconazole nitrate.

### BRIEF DESCRIPTION OF THE DRAWINGS

FIG. 1 is a graph which shows the dissolution rate of CAP film as a function of pH.

FIG. 2 is a graph which depicts the dependence of CAP solubility on pH.

FIG. 3 is a graph which shows that greater than 30 ml of blood per gram of CAP is required to bring the pH to levels at which the solubility of CAP starts to increase.

FIG. 4 is a graph which depicts the effect of anionic polymers on clotting of ACD plasma.

FIG. 5 is a graph which show the binding of HSV-2 to micronized CAP (Aquateric® (containing approximately 67wt % CAP)).

FIG. 6 is a graph which shows the pH changes by addition of seminal fluid to 180 mg of Aquateric®.

FIG. 7A is a cross-sectional view of the dual-compartment applicator device depicted in FIG. 1 of German patent application DE 20 2005 00 4 135 U1.

FIG. 7B is a top view of the dual-compartment applicator device depicted in FIG. 1 of German patent application DE 20 2005 00 4 135U1.

FIGS. 8A and 8B are graphs which show that CAP suppresses S. Aureus growth and toxic shock syndrome toxin 1 production.

### DETAILED DESCRIPTION OF THE INVENTION

The present invention relates to the utilization of an insoluble micronized form of cellulose acetate phthalate or a solid dosage form of cellulose acetate phthalate as an active ingredient, such as in anti-infective/feminine hygiene products which can be employed for long-term repeated use. The present invention concerns the use of micronized cellulose acetate phthalate to maintain a low vaginal pH, and the combined use of micronized CAP and Lactobacilli to maintain a normal acidic vaginal environment. The present invention also provides for the utilization of micronized cellulose acetate phthalate to prevent the propagation of Staphylococcus aureus and toxic shock syndrome toxin 1 during menstruation. The present invention also employs micronized cellulose acetate phthalate to prevent the shedding and dissemination of herpesvirus and to treat herpes lesions. The present invention also utilizes micronized cellulose acetate phthalate in combination with miconazole nitrate to treat Candida albicans infections.

The effectiveness of the active ingredient of the present invention (CAP) requires only a brief contact time between human tissue and the active ingredient. Such application is possible due to the safety of CAP, especially in its micronized form, and its low pH buffering capacity in the presence of physiological fluids and its exceedingly large adsorptive capacity per weight unit.

The safety of soluble and micronized forms of CAP has been heretofore established in detail as shown in published papers and safety data from the manufacturers of CAP and the micronized form of CAP (Aquateric®), respectively (Eastman Chemical Company, Kingsport, Tenn.; FMC Corporation, Philadelphia, Pa.).

Single and Repeat Dose Toxicity and Carcinogenicity Studies Single Dose Toxicity Studies

CAP can be found in the Inactive Ingredient Guide, where it is defined as an approved drug excipient currently marketed for human use for oral dosage forms. CAP safety has been extensively studied and it has been shown to be free of adverse effects (Neurath A R, Strick N, Li Y Y, Lin K, Jiang S, “Design of a ‘microbicide’ for prevention of sexually transmitted diseases using ‘inactive’ pharmaceutical excipients,’ Biologicals, 17:11-21 (1999)).

FMC Corporation (Philadelphia, Pa.) (U.S. Pharmacopeial Convention, Inc. The U.S. Pharmacopeia; pp. 780-781, (2000)) has performed extensive toxicity testing on micronized form of CAP, i.e., Aquateric® (containing 66-73 wt. % micronized CAP, a polyoxyethylene-polyoxypropylene block copolymer and distilled acetylated monoglycerides). The following Tables 1 and 2 contain toxicological information on Aquateric® from the FMC Corporation Material Safety Data Sheet.

TABLE 1
Toxicological Information for Aquateric ®

| | | Animal |
|---|---|---|
| Type of Study | Results | Mode |
| Eye Irritation | Non-irritating | Rabbits |
| Dermal Irritation | Non-irritating | Rabbits |
| Dermal Sensitization | Non-sensitizing | Guinea Pig |
| Skin Absorption | Dermal LD₅₀ >2,000 mg/kg | Rabbit |
| Inhalation | LC₅₀ >5.21 mg/L/4 hr (maximum | Rat |
| | attainable concentration, | |
| | no mortalities) | |
| Ingestion | Oral LD₅₀ >5,000 mg/kg | Rat |

TABLE 2
Summary of CAP Single Dose Toxicity Studies (from FMC Corporation)

| Species | | Group | | | | |
|---|---|---|---|---|---|---|
| (Strain) | Route | Size | Test Article | Dose/Duration | Results | Reference |
| Rat | Oral | 5M + 5F | 66-73 wt. % | 5,000 mg/kg/ | There were no deaths during the 14-day observation period. | FMC Corporation |
| (Sprague- | | | CAP in | single oral dose | The only clinical signs observed were oral discharge in one | study #: I83-796 |
| Dawley) | | | Aquateric ® | | female rat and diarrhea in one male rat. Aquateric ® CD-910 | |
| | | | CD-910 | | is classified as practically non-toxic (the LD₅₀ is greater than | |
| | | | | | 5,000 mg/kg | |
| Rat | Inha- | 5M + 5F | 66-73 wt. % | gravimetric | During the 15-day observation period, there were no deaths | FMC Corporation |
| (not | lation | | CAP in | concentration of | and no signs of toxicity among the test animals. Irregular | study #: I83-800 |
| specified) | | | Aquateric ® | 5.21 mg/l/4 h | breathing and poor coat quality were observed among the test | |
| | | | CD-910 | | animals during the exposure. No gross lesions were found at | |
| | | | | | necropsy. Aquateric ® CD-910 is considered practically non- | |
| | | | | | toxic (the LD₅₀ is greater than 5.21 mg/l). | |
| Guinea | Topical | 10M + | 66-73 wt. % | 0.30 g (solid)/6 h | Aquateric ® CD-910 is judged to be non-sensitizing when | FMC Corporation |
| Pig | | 10F | CAP in | three induction | topically applied to Hartley guinea pigs. No responses were | study #: I92-1266 |
| (Hartley) | | | Aquateric ® | treatments one | noted among test animals following either the induction or | |
| | | | CD-910 | week apart | challenge application. No irritation was noted among any of | |
| | | | | | the challenge control guinea pigs during challenge. Animals | |
| | | | | | in the positive control group exhibited definite sensitizing | |
| | | | | | reactions following the challenge application. | |
| Rabbit | Topical | 5M + 5F | 66-73 wt. % | 2,000 mg/kg/24 h | Aquateric ® CD-910 is classified as practically non-toxic (the | FMC Corporation |
| (New | | | CAP in | | LD₅₀ is greater than 2000 mg/kg). There were no deaths | study #: I83-797 |
| Zealand | | | Aquateric ® | | during the 14-day observation period. No skin irritation was | |
| White) | | | CD-910 | | observed in any rabbit during the study. One rabbit had nasal | |
| | | | | | discharge and one rabbit had lacrimation. All but two rabbits | |
| | | | | | lost weight. | |
| Rabbit | Topical | 6 animals | 66-73 wt. % | 0.50 g/4 h | Aquateric ® CD-910 is judged to be non-corrosive and non- | FMC Corporation |
| (New | | | CAP in | | irritating to intact skin when applied topically to New | study #: I83-799 |
| Zealand | | | Aquateric ® | | Zealand White rabbits. No dermal irritation or corrosion was | |
| White) | | | CD-910 | | observed on any animal during any of the scoring intervals. | |
| Rabbit | Ocular | 3 animals | 66-73 wt. % | 0.10 gm in the right | Aquateric ® CD-910 is considered non-irritating to both | FMC Corporation |
| (New | | in washed | CAP in | eye/washed group: | washed and unwashed eyes. One hour after dosing, slight | study #: I83-798 |
| Zealand | | group | Aquateric ® | eyes washed after | conjunctivitis was observed in 4 of 6 unwashed eyes and 1 of | |
| White) | | 6 animals | CD-910 | 20-30 sec. of | 3 washed eyes. At 24 hours, one of the unwashed eyes had | |
| | | in | | treatment | slight chemosis. All other eyes had returned to normal. | |
| | | unwashed | | 0.10 gm in the right | | |
| | | group) | | eye/unwashed | | |
| | | | | group: eyes | | |
| | | | | remained unwashed | | |

### Repeat Dose Toxicity and Carcinogenicity Studies

Rat (Oral Study)

Kotkoskie et al. (Kotkoskie L A, Freeman C, Palmieri M A, “Subchronic toxicity and developmental toxicity studies in rats with Aquateric® aqueous enteric coating,” Internat. J. Toxicology, 18:109-116 (1999)) examined the subchronic toxicity of Aquateric® (containing 66-73% micronized Cellulose Acetate 1,2-Benzenedicarboxylate (Cellulose Acetate Phthalate: CAP)) in four groups of twenty male and twenty female rats fed 0, 5,000, 25,000, or 50,000 ppm of Aquateric® daily for 90 days. No deaths occurred during the study and no treatment-related, clinical signs were noted. Clinical chemistry investigations yielded no toxicologically significant findings and all incidental findings were within physiologically acceptable historical reference ranges. There were likewise no treatment-related effects on organ weights or organ- to body-weight ratios. Based upon these study results, the No-Observed-Adverse-Effect-Level [NOAEL; greatest concentration of amount of a substance found by experiment or observation which causes no detectable adverse alteration of morphology, functional capacity, growth, development or life-span of the target organism under defined conditions of exposure; IUPAC Compendium of Chemical Terminology, 2^{nd} Edition 1997, 65:2076 (1993), http://www.iupac.org/goldbook/N04208.pdfl exceeds 50,000 ppm Aquateric® daily in the diet. This represents an average dosage of 3,604 or 4,094 mg/kg/day for male and female rats, respectively, which is approximately 200 times the anticipated clinical topical dose of Aquateric® used as a microbicide. In a chronic oral dosing experiment by Hodge (Hodge H, “The chronic toxicity of cellulose acetate phthalate in rats and dogs,” J. Pharmacol. Exp. Therapeutics, 80, 250-255 (1944)), four groups of 20 female rats each, were fed 0, 5, 20 and 30% CAP, ad libitum, for one year. The diet consisted of a Purina fox chow meal into which CAP was mixed and given ad libitum. The rats on high intake of CAP showed a reduction in growth rate, which increased with the dosage. No abnormalities were observed during autopsy. Histological examinations showed no consistent pathological changes. In general, no toxic effects of CAP have been found in rats.

Dog (Oral Study)

Three groups of 2 dogs each were fed 1, 4 or 16 μm of CAP daily for one year. The dogs remained in excellent health and condition throughout the experiment and no consistent pathological changes were discovered at autopsy. There was no evidence of toxic effects related to CAP in this study (Hodge H, “The chronic toxicity of cellulose acetate phthalate in rats and dogs,” J. Pharmacol. Exp. Therapeutics, 80, 250-255 (1944)). Feeding of CAP to rats or dogs for one year showed no evidence of target organ toxicity. In the subchronic study, groups rats received 0 (control), 5,000, 25,000, or 50,000 ppm (dose-range of 3600 to 4100 mg/kg/day) Aquateric® (containing 67% CAP) in the diet for 90 consecutive days. No mortality, clinical signs of toxicity or adverse toxicological effects on hematology or serum chemistry parameters, body weights, feed consumption, ophthalmological examinations, or histological evaluation of tissues were noted in any treatment group.

The following Table 3 is a summary of CAP repeat dose toxicity and carcinogenicity studies:

TABLE 3
Summary of CAP Repeat Dose Toxicity and Carcinogenicity Studies (from Literature)

| Species | | Group | Test | | Dose/ | | |
|---|---|---|---|---|---|---|---|
| (Strain) | Route | Size | Article | Formulation | Duration | Results | Reference |
| Rat | Oral | 20F | CAP | CAP mixed | 0, 5, 20 30% in | No toxic action of CAP was found in rats. The rats on | Hodge, 1944 |
| (albino) | | | | with food | food ad libitum | high intakes of CAP showed a reduction in growth rate, | |
| | | | | | for 12 months. | which increased with the dosage. On autopsy, the rats | |
| | | | | | | were in good condition and no abnormalities were | |
| | | | | | | observed except that the average stomach weight tended | |
| | | | | | | to increase with higher doses of CAP. From | |
| | | | | | | histological examination, no consistent pathological | |
| | | | | | | changes were demonstrated. NOAEL >30% in food | |
| Rat | Oral | 20M + 20F | 66-73 wt. % | Aquateric ® | 0, 5,000, | In a subchronic toxicity study no treatment-related | Kotkoskie |
| (Sprague- | | per group | CAP in | mixed with | 25,000, 50,000 | deaths, hematological finding or clinical signs during | et al., 1999 |
| Dawley) | | | Aquateric ® | food | ppm in food | the study. No toxicologically significant findings in the | |
| | | | | | for 90 days | clinical chemistry. No effect on body weights at 5,000 | |
| | | | | | | or 25,000 ppm Aquateric diet. Body weights were | |
| | | | | | | significantly reduced at 50,000 Aquateric diet | |
| | | | | | | investigations. NOAEL 25,000 ppm | |
| Dog | Oral | 6 animals | CAP | CAP mixed | 1, 4, 16 gm in | No evidence of any toxic effects of CAP. The dogs | Hodge, 1944 |
| (not | | | | with food | food/day for | remained in excellent health and condition throughout | |
| specified) | | | | | 12 months | the experiment and no consistent pathological changes | |
| | | | | | | were discovered at autopsy. NOAEL >16 gm | |

Mutagenicity

Batt and Kotko ski (Batt K J, Kotkoskie L A, “An evaluation of genotoxicity tests with Aquateric aqueous enteric coating”, Internat. J. Toxicology, 18:117-122 (1999)) looked at the mutagenic potential of Micronized CAP in the Ames test, a mouse lymphoma mutation assay, and in a mouse micronucleus test. Results of all three tests were negative, suggesting that Micronized CAP is not mutagenic or genotoxic in this standard battery of tests (see the following Table 4).

TABLE 4
Summary of CAP Genotoxicity Studies (from Literature)

| Species | | | | | | |
|---|---|---|---|---|---|---|
| (Strain) | Route | Test Article | Formulation | Dose/Duration | Results | Reference |
| Mice | Oral | 66-73 wt. % | Aquateric ® | 7,200 mg/kg/single | Mouse Micronucleus Assay: During the mouse | Batt and |
| (CD-1) | | CAP in | | dose | micronucleus assay all animals appeared normal after | Kotkoskie, |
| | | Aquateric ® | | | dosing and remained healthy until the end of the study. | 1999 |
| | | | | | Aquateric ® did not induce significant increases in | |
| | | | | | micronucleated polychromatic erythrocytes when | |
| | | | | | compared to the vehicle controls in either male or female | |
| | | | | | mice at any of the harvest times. NOAEL >7,200 mg/kg | |
| In Vitro | In | 66-73 wt. % | Aquateric ® | 0, 50, 167, 500, 1,667, | Ames Test: Aquateric ® was not toxic to the Salmonella | Batt and |
| (Salmonella | Vitro | CAP in | solubilized in | 5,000 μg/plate in 50 μl | tester strains (TA1535, TA1537, TA1538, TA98, TA100). | Kotkoskie, |
| typhimurium) | | Aquateric ® | dimethyl- | DMSO/incubation at | At the maximum dose, the test material did not cause an | 1999 |
| | | | sulfoxide | 37° C. for 48 h | increase in the mean number of revertants compared to the | |
| | | | (DSMO) | | solvent control with or without metabolic activation by | |
| | | | | | Aroclor-induced rat liver microsomes in any of the tester | |
| | | | | | strains. NOAEL >5,000 μg/plate in 50 μl DMSO | |
| In Vitro | In | 66-73 wt. % | Aquateric ® in | 116, 231, 750, 923, | Mouse Lymphoma Assay in the absence of metabolic | Batt and |
| (L5178Y | Vitro | CAP in | deionized water | 1,500, 2,000 μg/ml in | activity: The test material was lethal at 2,500 μg/ml and | Kotkoskie, |
| TK+/− mouse | | Aquateric ® | | the absence of | above. Treatment conditions were highly toxic at the | 1999 |
| lymphoma | | | | metabolic | 2,000 μg/ml dose with moderate to no toxicity at the lower | |
| cells) | | | | activation/incubation | concentrations. Mutant frequencies for the treated cultures | |
| | | | | at 37° C. for 4 hours | ranged from 31.3 × 10⁻⁶-55.0 × 10⁻⁶ and did not meet the | |
| | | | | with 2-day recovery | criteria for a positive or mutagenic response. | |
| | | | | and expression period | NOAEL >1,500 μg/ml | |
| In Vitro | In | 66-73 wt. % | Aquateric ® in | 116, 231, 554, 738, | Mouse Lymphoma Assay in the presence of metabolic | Batt and |
| (L5178Y | Vitro | CAP in | deionized water | 923, 1,250 μg/ml in | activity: At 1,250 μg/ml, there was relative growth with | Kotkoskie, |
| TK+/− mouse | | Aquateric ® | | the presence of | moderate to no toxicity at the remaining concentrations. | 1999 |
| lymphoma | | | | metabolic | All doses above 1,250 μg/ml were lethal to the cells. None | |
| cells) | | | | activation/incubation | of the cultures treated with the test material had mutant | |
| | | | | at 37° C. for 4 hours | frequencies which exceeded the minimum criteria for a | |
| | | | | with 2-day recovery | positive or mutagenic response. Mutant frequencies of the | |
| | | | | and expression period | treated cultures ranged from 36.3 × 10⁻⁶-58.0 × 10⁻⁶. | |
| | | | | | NOAEL >1,250 μg/ml | |

Reproductive and Developmental Toxicity

Kotkoskie et al. (Kotkoskie L A, Freeman C, Palmieri M A, “Subchronic toxicity and developmental toxicity studies in rats with Aquateric® aqueous enteric coating,” Internat. J. Toxicology, 18:109-116 (1999)) examined developmental toxicity of Micronized CAP in rats. Groups of 25 pregnant Sprague-Dawley rats received 0, 5,000, 25,000, or 50,000 ppm of Micronized CAP in their diet ad libitum on days 6 through 15 of gestation. Upon sacrifice at day 20, no deaths and no significant differences in body weights or gravid uterine weights were observed. In addition there were no treatment-related, significant differences in Caesarean section parameters or observed gross lesions. Only one fetal malformation was noted (micrognathia), which was considered spurious and unrelated to treatment. There were no fetal external variations and no statistically significant differences in the fetal or litter incidences of visceral or skeletal variations.

Kotkoskie et al. (Kotkoskie L A, Freeman C, Palmieri M A, “Subchronic toxicity and developmental toxicity studies in rats with Aquateric® aqueous enteric coating,” Internat. J. Toxicology, 18:109-116 (1999)) also examined subchronic toxicity in 20 male Sprague-Dawley CD rats. Rats were administered Micronized CAP in diet at concentrations of 0, 5,000, 25,000, or 50,000 ppm for 90 consecutive days. Males receiving 50,000 ppm Micronized CAP had decreased absolute testicular weights; however, relative testicular weights (testes to brain weight ratios) were unaffected. No histological alterations were present that correlated with the decrease in absolute testes weight.

The following Table 5 is a summary of CAP reproductive toxicity studies:

TABLE 5
Summary of CAP Reproductive Toxicity Studies (from Literature)

| Species | | | Test | | | | |
|---|---|---|---|---|---|---|---|
| (Strain) | Route | Group Size | Article | Formulation | Dose/Duration | Results | Reference |
| Rat | Oral | 25 F/group | 66-73 wt. % | Aquateric ® | 0, 5,000, 25,000, 50,000 ppm | No treatment-related maternal or | Kotkoskie |
| (Sprague- | | (pregnant) | CAP in | mixed with food | in food/from day | developmental fetal effects were | et al., |
| Dawley) | | | Aquateric ® | | 6 through day 15 of | observed. No fetal external variations | 1999 |
| | | | | | gestation | or visceral malformations were noted in | |
| | | | | | | any fetus examined. NOAEL >50,000 | |
| Rat | Oral | 20 M/group | 66-73 wt. % | Aquateric ® | 0, 5,000, 25,000, 50,000 ppm | Males receiving 50,000 ppm | Kotkoskie |
| (Sprague- | | | CAP in | mixed with food | in food/90 days | Aquateric ® had decreased absolute | et al., |
| Dawley) | | | Aquateric ® | | | testicular weights; however, relative | 1999 |
| | | | | | | testicular weights (testes to brain weight | |
| | | | | | | ratios) were unaffected. No histological | |
| | | | | | | alterations were present that correlated | |
| | | | | | | with the decrease in absolute testes | |
| | | | | | | weight. | |

Local and Photo-Sensitization
Guinea Pig (Dermal Study)

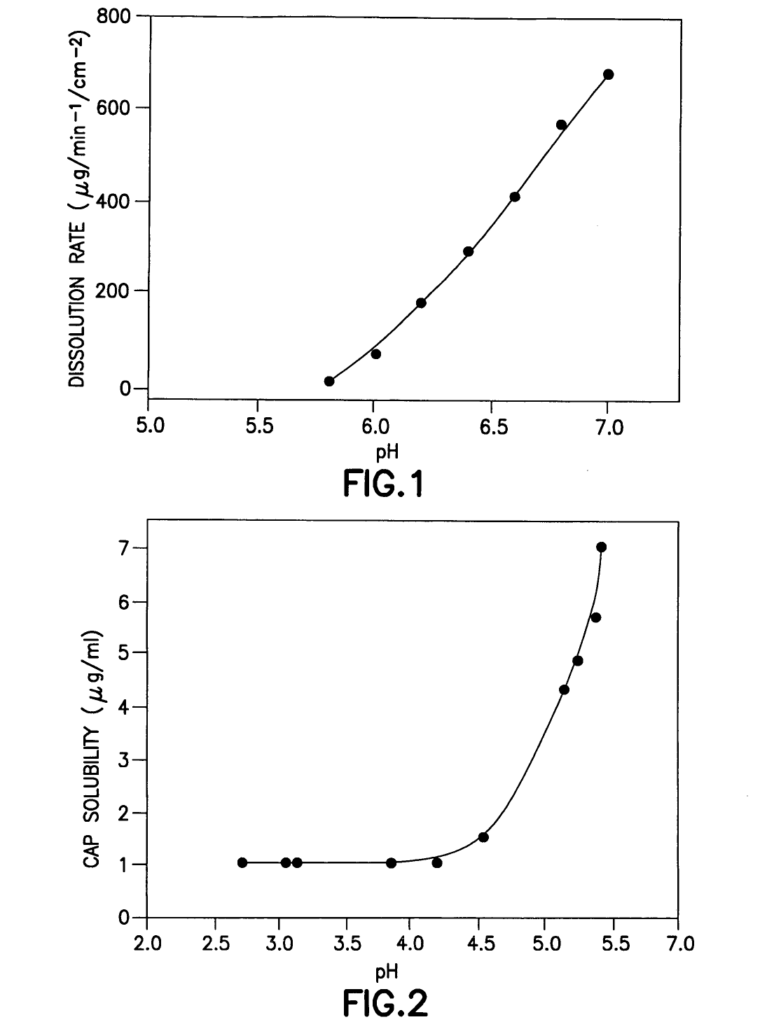

The manufacturer of Aquateric® CD-910 (containing 66-73wt. % micronized Cellulose Acetate 1,2-Benzenedicarboxylate (Cellulose Acetate Phthalate: CAP)) (FMC Corporation) performed a skin sensitization study with subsequent induction treatment on Hartley guinea pigs. After three induction treatments one week apart to skin treated with Aquateric®, it was determined that Aquateric® was non-sensitizing when topically applied to Hartley guinea pigs. No responses were noted among test animals following either the induction or challenge application. No irritation was noted among any of the challenge control guinea pigs during challenge. Animals in the positive control group exhibited definite sensitizing reactions following the challenge application. The solubility of CAP is reasonably high at pH 7 and above. CAP is only minimally soluble at pH 6; below pH 6 the solubility further decreases (see FIG. 1). More detailed studies carried out by using a new newly developed sensitive method for the spectrophotometric determination of CAP (Neurath A R, Strick N, “Quantitation of cellulose acetate phthalate in biological fluids as a complex with ruthenium red,” Anal. Biochem, 288:102-04 (2001)) revealed that the solubility of CAP is approximately 7 μg/ml at pH 5.5 and further decreases with decreasing pH (see FIG. 2). The NOAEL from a 90-day study in rats (Kotkoskie L A, Freeman C, Palmieri M A, “Subchronic toxicity and developmental toxicity studies in rats with Aquateric® aqueous enteric coating,” Intemat. J. Toxicology, 18:109-116 (1999)) was approximately 2, 450 mg soluble CAP/kg/day. This safe dose of CAP is practically unachievable if an insoluble micronized form of CAP is used, provided that the environment is kept at pH levels <5.5 (see FIG. 2).

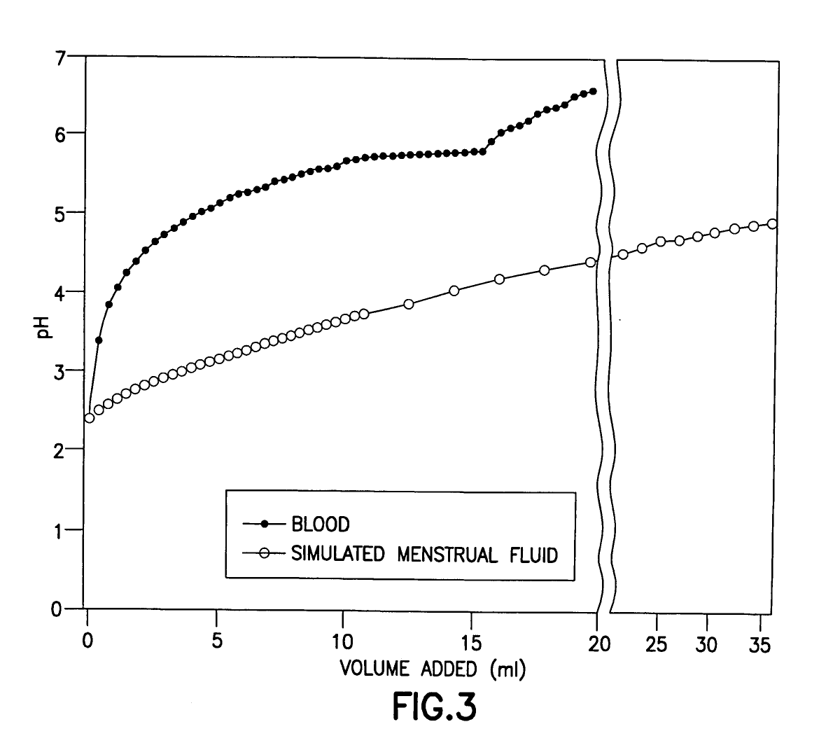

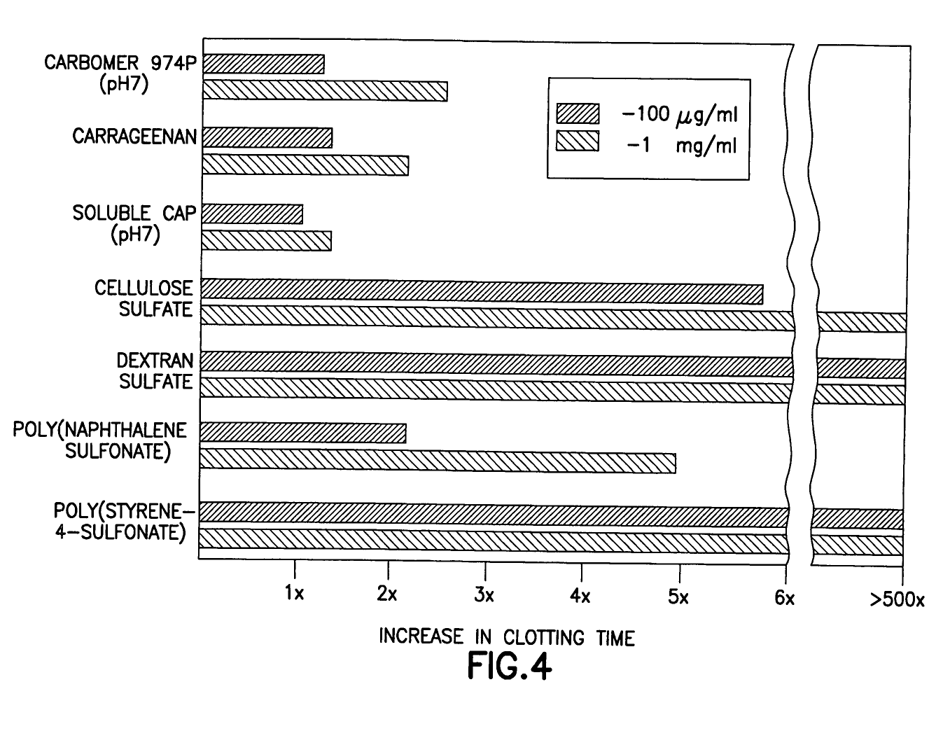

The inventors' experiments have demonstrated the surprisingly high buffering capacity at low pH of micronized CAP. Results shown in FIG. 3 indicate that >30 ml of blood per gram of CAP is required to bring the pH to levels at which the solubility of CAP starts to increase (to >7 μg/ml). The volume of menstrual fluid needed per gram of CAP would be much higher than that required for blood (FIG. 3). 448 mg of CAP in the form of a water dispersible film (Neurath A R, Strick N, Li Y Y, “Water dispersible microbicidal cellulose acetate phthalate film,” BMC Infect. Dis., 3:27 (2003), http://www.biomedcentral.com/content/pdf/1471-2334-3-27.pdf; Neurath A R, Strick, Li Y Y, ‘Water dispersible film,” US Pat. Application Publication No. 2005/0070501 A1 (published Mar. 21, 2005)) was used in these experiments. Rabbit blood was used in these experiments which has a similar buffering capacity to human blood. The stimulated menstrual fluid was prepared as described in Geshnizgani A M, Onderdonk A B, “Defined medium simulating genital tract secretions for growth of vaginal microflora,” J. Clin. Microbiol., 30:1323-132, (1992)).

CAP can safely be used in many physiological environments in which it is in micronized form. Due to its high buffering capacity, CAP will provide a low pH. This new finding is essential for the application of micronized CAP, in distinct forms and formulations, as an anti-infective/general hygiene product.

The safety of micronized CAP was further established as follows. A 14-day rabbit irritation study was conducted, in which 1 ml of formulations containing 130 mg of micronized CAP were applied daily vaginally to rabbits. These studies established that CAP at the concentrations and volumes used may be considered acceptable for human use. In contrast, treatment of rabbits with “CONCEPTROL” vaginal gel, a commercially available vaginal contraceptive product, resulted in vaginal irritation in all rabbits, that would be considered borderline or unacceptable for human use.

A gel formulation of micronized CAP (130 mg/g) was also applied vaginally to rhesus monkeys. Serum chemistries, vaginal biopsies, bacterial cultures and vaginal pH were determined to be within normal limits after dosing with CAP formulations. No obvious changes in peripheral CD4:CD8 cell ratios or levels of inflammatory cytokines/chemokines in plasma and vaginal fluids were detected. Colposcopy examinations determined that CAP formulations were not irritating (Boadi T, Schneider E, Chung S, Tsai L, Gettie A, Ratterree M, Blanchard J, Neurath A R, Cheng-Mayer C, “Cellulose acetate 1,2-benzenedicarboxylate protects against challenge with pathogenic X4 and R5 simian-human immunodeficiency viruses,” (submitted to AIDS Jan. 27, 2005)).

Biocompatibility of CAP Film with Human Vaginal Epithelia

The potential toxicity of anti-infective/hygiene products, respectively is usually evaluated in vitro using dilutions of the active ingredient or of the final product. Undiluted formulations are usually not tested in this way. New methods for evaluation of undiluted formulations have been developed by Dr. Raina N. Fichorova from the Brigham and Women's Hospital, Boston, Mass.

The disruption of the epithelial barrier and activation of inflammatory pathways may lead to increased susceptibility to viral infection. Over-the-counter (OTC) vaginal products have received little attention in this regard. However, the recent development of new vaginal products has brought the importance of mucosal integrity to forefront. They can be efficacious only if they cause no epithelial toxicity of inflammation. A novel organotypic cervicovaginal model (VEC100; MatTek Corp., Ashland, Mass.) was used to compare hygiene or spermicidal over the counter (“OTC”) products to CAP. CAP film and gel formulations appeared non-toxic in MTT and LDH viability assays and did not induce release of pro-inflammatory mediators, e.g., IL-1, IL-6 and IL-8 as compared to controls. In contrast, a commercially available vaginal cleansing film containing Nonoxynol-9 (Apothecus Pharmaceutical Corporation, Oyster Bay, N.Y.) were proinflammatory. This study demonstrated the usefulness of the VEC tissue model for immunoinflammatory characterization and safety assessment of vaginal products. It provides evidence that CAP in film and gel form may be used as a safe vaginal product (Trifonova R T, Pasicznyk J M, Fichorova R N, “Vaginal formulations—how innocuous are they to the vaginal epithelial immune function?” American Journal of Reproductive Immunology, 25^{th} Anniversary Meeting, Jun. 16-18, 2005, Providence, R.I., (2005)).

In agreement with the rabbit vaginal irritation results, these new results further confirm the lack of toxicity of micronized CAP and CAP film as compared with some of the OTC products which appeared much less safe and more toxic than the formulations of micronized CAP.

Another unique property of micronized CAP (pH <6.0) is the very large surface area per unit weight (see the following Table 6).

TABLE 6
Colloidal Chemistry of CAP/Aquateric* Formulations

| | |
|---|---|
| Number of micronized particles per | ≈1.5 × 10¹² |
| gram Aquateric ® | |
| Number of micronized particles per unit | ≈0.68 × 10¹² |
| dose (2.5 g) of formulated | |
| CAP/Aquateric ® (18 wt. % Aquateric ®) | |
| Total surface area for 1 gram | ≈5.1 m² |
| Aquateric ® | |
| Total surface of micronized particles in | ≈2.3 m² |
| one unit dose (2.5 g) of formulated | |
| CAP/Aquateric ® | |

*Aquateric ® is a micronized form of CAP (FMC Corporation, Philadelphia, PA) containing ˜67 wt. % of CAP

The total surface area was calculated on the assumption that the surface of these particles is smooth. Indentations on the surface would increase the surface area of the particles. This indicates that the micronized CAP particles are expected to have a high adsorptive capacity for viruses and/or microorganisms.

Since heparin and other sulfated polymers are known to inhibit blood clotting (de Raucourt E, Mauray S, Chaubet F, Maiga-Revel O, Jozefowicz M, Fischer A M, “Anticoagulant activity of dextran derivatives,” J. Biomed. Mater. Res., 41:49-57 (1998)), the effect of sulfated and carboxylated polymers, respectively, on blood clotting was measured. The final concentrations of the compounds tested were 100 μg/ml and 1 mg/ml (see FIG. 4). It is evident from these results that soluble CAP, unlike any of the other polymers, did not increase clotting time, and therefore is not expected to prolong the duration of bleeding. In this respect, since micronized CAP is being used, the level of soluble CAP will be at least 2 orders of magnitude lower than the level tested in these experiments (=1 mg/ml). These results are important for applying micronized CAP as an anti-infective/hygiene product. Thus, applications to sites at which bleeding might occasionally occur (wound healing) or where bleeding is an integral part of a physiological process (menstruation) is possible and not contraindicated.

In an embodiment of the invention, a tampon has insoluble micronized cellulose acetate phthalate powder being incorporated thereon (or therein). Alternatively, the tampon could be wrapped in a cellulose acetate phthalate film.

A tampon is an absorbent member primarily designed to be worn by a woman during her menstrual period to absorb menses, blood and other body fluid. The tampon includes a tampon body and a withdrawal string. The tampon body is normally compressed into the form of a cylinder and can have a blunt, rounded or shaped forward end. The tampon body has a forward or distal end that is closer to the cervix when the tampon is in use. The tampon body also has a proximal end that is closer to the vaginal opening when the tampon is in use.

The tampon commonly has a withdrawal string fastened to the proximal end that serves as a means for withdrawing the tampon from the woman's vagina. The withdrawal string can be looped through an aperture formed transversely through the tampon body. In addition, the withdrawal string can have a knot formed at the free end of the string to assure that the string will not separate from the tampon body.

Catamenial tampons suitable for use in the present invention include an absorbent. The absorbent can be formed from fibers that are assembled into an absorbent sheet or ribbon. Alternatively, the absorbent can be formed from absorbent fibers that are assembled and compressed into a generally elongated and/or cylindrical configuration. The absorbent is desirably formed from cellulosic fibers, such as cotton and rayon. For example, the absorbent can be 100% cotton, 100% rayon, a blend of cotton and rayon fibers, or other materials known to be suitable for tampons, including artificial fibers such as polyester, polypropylene, nylon or blends thereof. The absorbent may also include degradable fibers. Other types of materials or structures may also be used, such as cellulose sponge or a sponge formed from elastomeric materials. When formed, the absorbent typically includes interstitial space or voids between the fibers or other materials.

Tampons suitable for use in this invention are usually made of absorbent fibers, including one or both of natural and synthetic fibers, compressed into a unitary body of a size that may easily be inserted into the vaginal cavity. Fiber orientation is typically in a linearly- or radially-wound structure. Tampons are normally made in an elongated cylindrical form in order that they may have a sufficiently large body of material to provide the required absorbing capacity, but may be made in a variety of shapes. The tampon is typically compressed. Compression may be achieved by predominantly axially- or radially-applied pressure. The tampon may be made of various fiber blends including both absorbent and nonabsorbent fibers, which may or may not have a suitable cover or wrapper. The cover or wrapper for absorbent products, such as tampons and sanitary napkins, is often made from a sheet of spunbonded fibers, e.g., a spunbond polypropylene sheet. The tampon may also include one or more of various treatments to improve the performance of the tampon, including reduced friction and increased absorption, delivery of the therapeutic agent, or both.

Tampons are further described in U.S. Pat. No. 6,860,874; U.S. Pat. No. 6,888,043 and U.S. Pat. No. 7,087,045, the entire contents of all of which are hereby incorporated by reference herein.

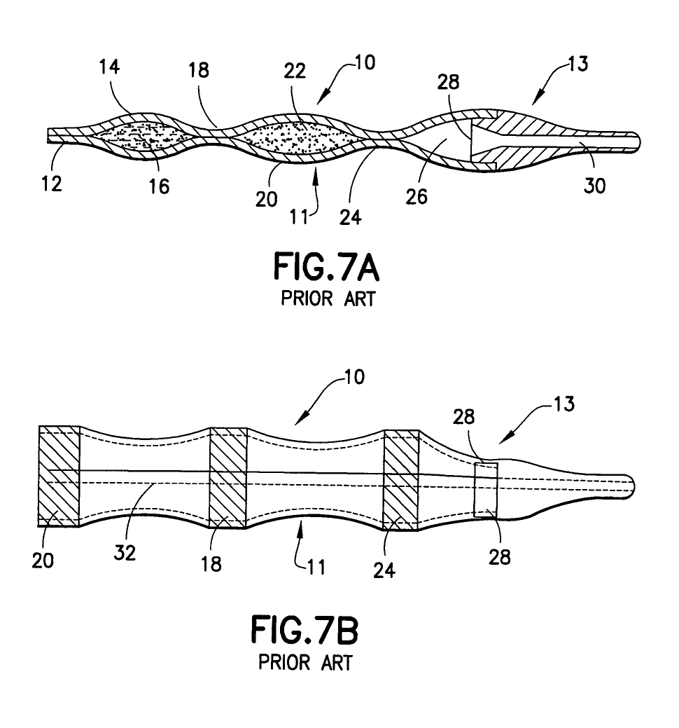

Distinct formulations of CAP can be used for the indications discussed herein. This includes CAP films (Neurath A R, Strick N, Li Y Y, “Water dispersible microbicidal cellulose acetate phthalate film,” BMC Infect. Dis., 3:27 (2003), http://www.biomedcentral.com/content/pdf/1471-2334-3-27.pdf; Neurath A R, Strick N, Li Y Y, “Water dispersible film,” US Pat. Application Publication No. 2005/0070501 A1 (published Mar. 21, 2005), the entire contents of which are hereby incorporated by reference herein), combinations of CAP films with tampons specifically suitable for the suppression of S. aureus and TSST-1 production, CAP in the form of rapidly dispersing tablets, and CAP/Aquateric® gels which are delivered in a gel form after mixing Aquateric® powder with an inactive gel having bioadhesive properties, the two distinct components being separated from each other within a dual-compartment applicator, such as disclosed in German Patent Application DE 20 2005 004 135 U1 (WO 2006/094483), Assignee: Klocke Verpackungs-Service GmbH, Weingarten, Germany (see FIGS. 7A and 7B of this application)).

With reference to FIGS. 7A and 7B, wherein like reference numerals correspond to the same or similar elements, the dual compartment applicator device 10 is depicted. The dual compartment applicator device 10 comprises a tubular packing element 11 as the base unit and an applicator portion 13.

The tublar packing element 11 can be made of a flexible film (a part capable of being extruded in tubular form is also usable) of plastic, aluminum or a composite materials thereof, which film can be laminated or coated depending on the intended use.

The tubular packing element 11 can be formed by an overlap of the long sides of the film. The film can be coated to give it sealing capability on both sides thereof, thus permitting a permanent or else a peelable bond to be obtained at the contact faces depending on the particular parameters, such as heat or pressure, applied during overlapping or layering of the film.

The tubular packing element 11 can therefore be made from a single flat film, which is made into a tubular form by a permanently bonded region (seal 32) running in the main direction thereof.

The tubular packing element 11 is permanently bonded at the back end of the dual-compartment applicator device 10 by a seal 12.

The applicator portion 12, which is at the front end of the dual-compartment applicator device 10, has a continuous delivery (extrusion) channel 30, which is preferably centrally disposed in the device 10. The extrusion channel 30 forms a path to the reservoir 26 disposed at the front end region of the tubular packing element 11. A seal 28 is disposed at the end of the extrusion channel 30 which faces reservoir 26.

The rear end region of the tubular packing element 11 is permanently closed by seal 12. A gel chamber 14 containing a gel 16 is sealed at one end by the seal 12 and is detachably sealed at the opposite end by a detachable seal 18. The detachable seal 18 serves to separate the gel 16 from the powder 22 in a powder chamber 24, prior to the mixing of the gel 16 and the powder 22.

A detachable seal 24 is disposed between the powder chamber 20 and the reservoir 26. The detachable seal 24 forms a portion of the boundary of the powder chamber 20, thus ensuring against inadvertent escape of the powder 20.

To mix the gel 16 and the powder 22, pressure is exerted via the tubular packing element 11 on one of the components in the direction of the other component, whereby the detachable seal 20 between two components (the gel 16 and the powder 22) opens. The gel 16 and the powder 22 are mixed by squeezing the tubular packing element 11 alternately in the regions of said components.

By pressing or spreading out the mixed components further in the direction of the applicator portion 12, the detachable seal 24 is detached. The contents pass through the extrusion channel 30 of the applicator 13 to the outside and are deposited at the intended place by means of the applicator 10.

The reason for separating the Aquateric® powder from the water based gel during storage within the dual-compartment applicator is the fact that Aquateric® (CAP) is moisture sensitive and therefore cannot be provided in the form of a water containing gel.

In a preferred embodiment of the dual compartment applicator, the compartment nearest to the delivery application to be vaginally inserted contains the Aquateric® (e.g., 0.36 g), while the more distant compartment contains the bioadhesive gel (e.g., 1.64 g). The gel contains appropriate amounts of a gelling agent and water.

The gelling agent is at least one of hydroxyethylcellulose (“HEC”), hydroxypropylcellulose, hydroxypropylmethylcellulose, guar gum, gelatin, ethylcellulose, alginic acid, carrageenan, carboxymethylcellulose sodium, methylcellulose, poloxamer, polyethylene glycol, polymethacrylate, polyvinyl alcohol, propylene glycol alginate, sodium alginate, starch (pregelatinized), tragacanth or xanthan gum.

The gel can also contain an appropriate preservative to prevent bacterial contamination during storage, e.g., sorbic acid or parabens, at commonly used levels.

In the context of the present invention, a “gel” is defined as being of high viscosity produced by combining a solvent and a polymer.

The mixture of micronized CAP and the water-based gel has the ability to adsorb both HIV and herpesvirus completely, i.e., the virus binds to the composition.

The present invention is thus also directed to a method which involves using a dual compartment applicator for maintaining the micronized CAP powder in one compartment and maintaining the water-based gel in another compartment during storage and premixing the materials from each of the two compartments (such as by breaking a seal between the two compartments by finger pressure), before use.

The gel thus can be delivered from a dual compartment applicator in which CAP in micronized form is separated from a water-based gel during storage, and the contents of the two compartments are mixed before delivery. The mixed composition can be then vaginally administered to a human female to prevent the shedding and dissemination of herpesvirus, treat or prevent bacterial vaginosis , maintain a low vaginal pH, or decrease the frequency of transmission of HIV (HIV-1 or HIV-2), herpesvirus or human cytomegalovirus.

The bacterium associated with bacterial vaginosis may be Mycoplasma hominis, Mycoplasma capricolum, Mobiluncus curtisii or Prevotella corporis.

The above method serves to combine incompatible drugs. Thus, water-sensitive ingredients can be included in the micronized CAP compartment, and water-soluble ingredients or drugs can be included in the water-based gel compartment.

The above method can also be used to administer a combination of cellulose acetate phthalate in one compartment of the applicator and Lactobacilli in the other compartment of the applicator for introducing the combination into a woman's vagina to maintain an acid healthy vaginal environment. Alternatively, CAP and Lactobacilli can be in the same compartment separated from a water based gel in the other compartment.

The term micronized used herein refers to particles having a particle size of about 1 micron.

Cellulose acetate phthalate has no effect on vaginal yeast infections (Candida albicans). ((Neurath A R, Strick N, Li Y-Y, Lin K and Jiang S (1999) Design of a “microbicide” for prevention of sexually transmitted disease using “inactive” pharmaceutical excipients. Biologicals 27: 11-21). To overcome this problem, micronized cellulose acetate phthalate can be combined with miconazole nitrate (2 to 5wt %). The resulting composition serves as an ideal formulation to maintain vaginal health by providing a low pH, treating herpesvirus and vaginal or sexually transmitted bacterial infections (for example, syphilis, gonorrhea, chlamydia trachomatis, chancroid and bacterial vaginosis) blocking toxic shock syndrome toxin generation during menstruation, and treating Candida albicans infections.

Administration of the CAP compositions discussed herein is usually vaginally, but with respect to some aspects of the present invention, administration may be topical to the skin or the genitalia.

The amount (dosage) of the active ingredient (CAP) for use in the present invention will, in general, be less than 1,000 milligrams, preferably between 200 to 800 milligrams.

For the prevention of shedding and dissemination of herpesvirus according to the present invention, the CAP formulation can be applied as a gel or a film. For the regular (for example, daily) application of CAP to maintain a normal acidic vaginal environment, the CAP formulation can be a gel, film, dispersible tablet or a water dispersible sponge which is converted into gel following application thereof (see U.S. Pat. No. 6,572,875 and U.S. Pat. No. 6,596,297, the entire contents of each of said patents being incorporated by reference herein). For treating herpes lesions, a premoistened cellulose acetate phthalate film can be topically applied to the lesion and the locus thereof.

Mucoadhesive instantly dispersible (water dispersible) tablets can be prepared from a combination of an insoluble micronized form of cellulose acetate phthalate and the hydroxypropyl methylcellulose (“HPMC”), “PHARMABURST” (a quick dissolving delivery platform having a bulk density of 0.450, a tapped density of 0.536 and a Carr's index of 16.0% made by SPI Pharma).

The compositions for use according to the present invention may also contain other active ingredients, such as spermicides, antimicrobial agents, preservatives or anti-viral agents.

The pharmaceutical compositions for use in the present invention may also contain additives such as preservatives and fragrances. These additives may be present in any desired concentration. The concentrations of these additives will depend upon the desired properties, the agent to be released, the potency, the desired dosage, the dissolution times, etc.

### EXAMPLES

The present invention will now be described with reference to the following non-limiting examples.

### Example 1

### Binding of HSV-2 to Micronized CAP (Aquateric®)

In order to investigate the adsorptive capacity of micronized CAP (=Aquateric®), graded quantities of this material (0.74 to 180 mg/ml) were added to a suspension containing 10 μg/ml of purified herpes virus type 2 (HSV-2) (Advanced Biotechnologies, Inc., Columbia, Md.) in 0.14 M NaCl containing 100 μg/ml of each bovine serum albumin (BSA) and gelatin, and mixed for 5 minutes at 37° C. The mixtures were put on ice, centrifuged at 10,000 rpm and the supernatant fluids were filtered through 0.45 μ filters which had been prewashed with 0.14 M NaCl containing 100 μg/ml of each BSA and gelatin. Dilutions of the supernatant fluid as well as dilutions of the original virus suspension were tested for the content of HSV-2 by an enzyme linked immunosorbent assay (ELISA). The dilutions were made in TRIS buffered saline (TBS) containing 1 wt/% of BSA and 0.25 wt. % of gelatin and 0.1 wt. % of TWEEN-20 at pH 7.2. The dilutions were added to wells of 96-well polystyrene plates coated with 100 ng of a monoclonal antibody to the HSV-2 envelope glycoprotein gE (Catalog # CC65901M; Biodesign International, Saco, Minn., USA). After overnight incubation at 25° C., the wells were washed 5 times with TBS containing 0.1 wt. % TWEEN-20 and 2,000-fold-diluted horse radish peroxidase (HRP) labeled antibody to HSV-2 (Catalog # B65124S; Biodesign International, Saco, Minn.) in 10 wt. % goat serum, 0.1 wt. % TWEEN-20 in TBS was added. After 2 hours at 37° C., the wells were washed, bound HRP was quantitated using a test kit from Kierkegaard and Perry Laboratories, Inc. (Gaithersburg, Md.) following the manufacturer's protocol, and the absorbance was read at 450 nm.

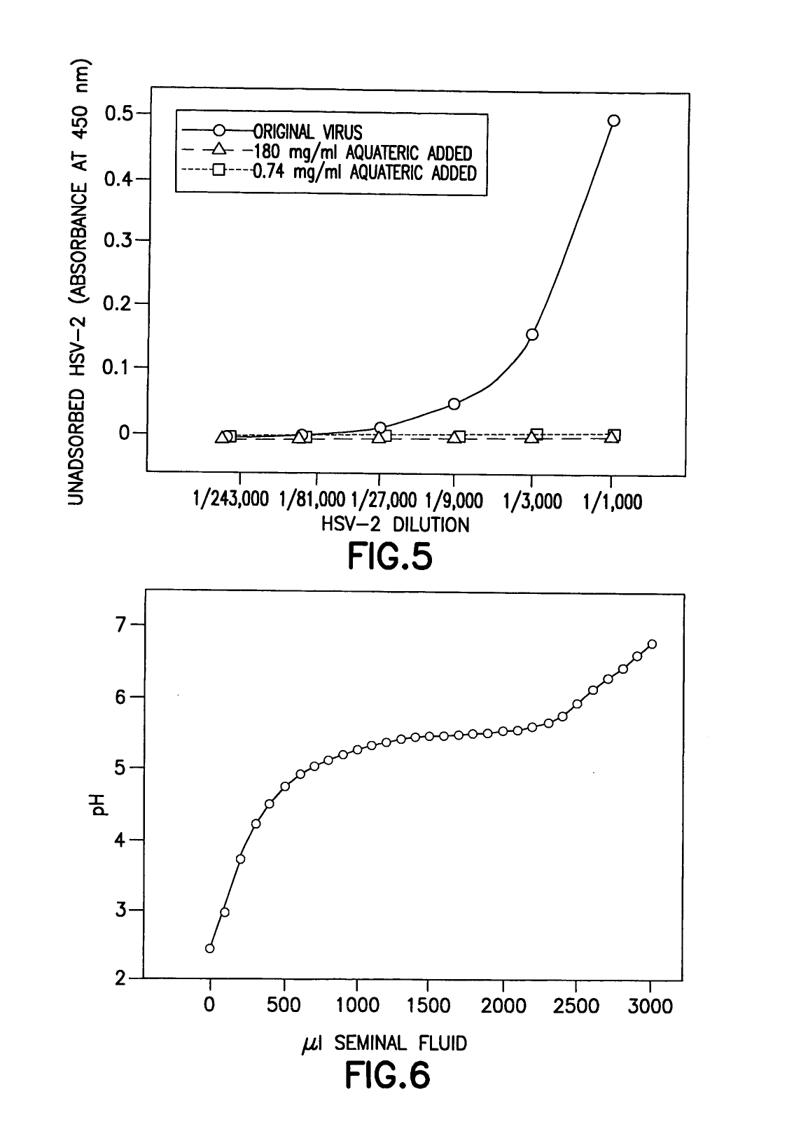

Results shown in FIG. 5 indicate that HSV-2 was completely adsorbed to Aquateric® even at the lowest concentration used (0.74 mg.ml). Complete adsorption of HSV-2 was also observed in the presence of an excess of proteins, e.g., 62% seminal fluid at an Aquateric® concentration of 69 mg/ml (other concentrations were not tested; data not shown).

### Example 2

### Lactobacilli Combinations

Lactobacilli were grown in the presence of micronized CAP. Surprisingly and unexpectedly, the Lactobacilli do replicate in the presence of micronized CAP. Thus, micronized CAP not only, does not affect the viability of Lactobacilli, but also provides an environment in which Lactobacilli can grow. Based on this surprising finding, micronized CAP can be combined with Lactobacilli to generate products maintaining and augmenting health by allowing colonization of mucosal sites by beneficial Lactobacilli and suppressing colonization by undesirable bacteria, which are also inactivated by the presence of micronized CAP.

Recent studies using solely a gene-based procedure and polymerase chain reaction (PCR) amplification, it was possible to precisely identify microbes in the vaginal epithelium of individual women (Hyman R W, Fukushima M, Diamond L, Kumm J, Giudice L C, Davis, R W, “Microbes on the human vaginal epithelium,” Proceedings of the National Academy of Sciences of the USA, 102:7952-7957 (2005)). The results have demonstrated that Lactobacilli, known to be necessary for maintaining a healthy vaginal environment, were not present in all women and the Lactobacilli content varied between 0-100%. Forty percent of the healthy women in these studies had no Lactobacilli and instead the undesirable microorganisms Bifidobacterium, Gardnerella, Prevotella, Pseudomonas and Streptococci predominated. The absence of Lactobacilli in the presence of the latter microorganisms is associated with a less acidic vaginal environment which predisposes for additional bacterial and viral infections, including infection by HIV-1. To correct this undesirable condition among healthy women, simple and consistently used preventive measures are needed. This includes the administration of Lactobacilli and the maintenance of a low acidic pH by simple pharmaceutical interventions, which include the administration of CAP formulations on a regular basis, for example, a daily basis.

Physicochemical interactions between male and female genital secretions as a result of sexual intercourse can modulate subsequent sexual transmission of HIV-1 and other STD pathogens. A significant contributory factor to this is the fact that semen contains bases such as spermine and phosphoryl choline and other components which cause in vivo neutralization of the acidic cervicovaginal pH leading to an increased pH and a decrease in natural defenses again STD transmission (Tevi-Benissan C, Belec L, Levy M, Schneider-Fauveau V, Mohamed A S, Hallouin M C, Matta M, Gresenguet G, “In vivo semen-associated pH neutralizaiton of cervicovaginal secretions,” Clin. Diagnostic Laboratory Immunol., 4:367-374 (1997)). This effect can be relatively long-lasting. Amelioration of this condition can be accomplished by administration of CAP formulations since a micronized form of CAP provides a strong low pH buffering capacity when mixed with seminal fluid (see FIG. 6). In these experiments graded volumes of seminal fluid were added to a suspension containing 180 mg of Aquateric®. This amount of Aquateric® corresponds to 1 ml of an Aquateric® formulation and to about 50% of a proposed human dose. The results indicate that Aquateric® significantly contributes to maintaining an acidic pH environment.

### Example 3

### Prevention of Toxic Shock Syndrome Toxin 1 (TSST-1) Production

Applicants designed experiments to determine whether incorporation of micronized CAP, and specifically of a CAP based dispersible film (Neurath A R, Strick N, Li Y Y, “Water dispersible microbicidal cellulose acetate phthalate film,” BMC Infect. Dis., 3:27 (2003), http://www.biomedcentral.com/content/pdf/1471-2334-3-27.pdf, Neurath AR, Strick N, Li Y Y, “Water dispersible film,” US Pat. Application Publication No. 2005/0070501 A1 (published Mar. 21, 2005)), would result in tampons with improved properties inhibiting S. aureus and TSST-1 production. The corresponding experiments and their results are described below.

Commercially available OTC Tampax S+ and o.b. super tampons, respectively, wrapped into water dispersible CAP film (Neurath A R, Strick N, Li Y Y, “Water dispersible microbicidal cellulose acetate phthalate film,” BMC Infect. Dis., 3:27 (2003), http://www.biomedcentral.com/content/pdf/1471-2334-3-27.pdf; Neurath A R, Strick N, Li Y Y, “Water dispersible film,” US Pat. Application Publication No. 2005/0070501 A1 (published Mar. 21, 2005)) (1.12 g of film per tampon) were evaluated for the production of Staphylococcus aureus and the toxic shock syndrome 1 (TSST-1) by the tampon sac method (Reiser R F, Denzin L K, Bergdoll M S, “Effects of blood and different media on the production of toxic shock syndrome toxin 1 by Staphylococcus aureus in the tampon sac method,” J. Clin. Microbiol., 26:2672-2674 (1988)). The film wrapping was firmly attached to the tampon by sealing the edges with a small amount of ethyl alcohol. The original tampons and the tampons wrapped in the CAP film were stored at room temperature until the time of testing. Samples in quintuplicate were asceptically removed from their packaging and when necessary, transferred to an applicator. The weight of each tampon was recorded. All tests were performed at Toxin Technology, Inc., Sarasota, Fla. Toxin Technology standard operating procedures (SOP) were employed in all testing.

Tampon Sac Method: Dialysis tubing was inoculated with approximately 5×10⁵ colony forming units (CFU) per 0.1 ml of S. aureus strain FRI-1169 wild type, a yellow pigmented strain which produces TSST-1. The inoculum was made using 0.9wt. % saline (NaCl, pH unadjusted) washed S. aureus. After inoculation of the sacs, the test samples were ejected from the applicator into the dialysis sac. The sac containing the sample was then placed in a tube containing Brain Heart Infusion Broth and 1.5wt. % agar at 45° C. The test unit was then placed in a cold water bath to facilitate hardening of the agar. After the agar was set, the test units were transferred to a 37° C incubator for 18 to 20 hours.

After the incubation period, the samples were removed from the dialysis sac and transferred to a pre-weighed sterile Whirl-Pac® bag and reweighed to determine the weight gain. The samples sere then extracted with 0.9wt. % saline (NaCl, pH unadjusted) containing 0.5wt. % Tween-20 for toxin analysis and CFU estimation. The spent applicators were weighed at this time so that the true sample weight could be determined.

CFU Estimation: The CFU were determined using the Toxin Technology SOP for plate counting. Briefly, log to the base 10 dilutions were prepared from the extract and pour plated using plate count agar. The 10⁻⁷, 10⁻⁸ and 10⁻⁹ dilutions were tested, the plates were incubated at 37° C. for approximately 40 hours before the colonies could be visually counted. The appropriate plate was then counted (a plate with 20-200 visible colonies) and the number recorded.

TSST-1 Determination: Two milliliters of the extract of each sample were centrifuged in order to remove the cells and prepare the sample for the Enzyme Linked Immunosorbent Assay (ELISA). The samples were then frozen to preserve them for the assay the following day. The samples were thawed at room temperature on the day of analysis. The samples were subjected to the Toxin Technology SOP for ELISA. Briefly, the day before the assay 96 well polystyrene plates were coated with anti-TSST-1 capture antibody. On the day of the assay, the following steps were performed:
1. The coated plate was washed to remove excess capture antibody.
2. Appropriate dilutions were made of the thawed samples, 1: 1,000 and 1:10,000.
3. A standard curve was prepared from purified TSST-1 at the following concentrations: 0.32, 0.63, 1.25, 2.5, 5.0 and 10 ng/ml.
4. The diluted samples, the standards, and a negative control were tested in triplicate wells on the coated plate.
5. After incubation at 37° C., the plate was emptied and washed repeatedly.
6. The second anti-TSST-1 antibody conjugated to horseradish peroxidase was applied and incubated at 37° C.
7. The plate was then again emptied and washed repeatedly.
8. A substrate solution of ABTS® solution [2,2′-azinobis(3-ethylbenzthiazoline-6-sulfonic acid)] (Boehringer Mannheim, Germany) was applied and observed for color development.
9. After a 15 minute incubation, the reaction was stopped. The plate was read on an ELISA plate reader and the data was recorded.

Data Analysis:

Colony Forming Units: The CFU were determined by multiplying the reciprocal number of the appropriate dilution times the colony count, e.g., 71 Colonies of the 10⁻⁹ dilution plate equals 7.1×10¹⁰ CFU. Total CFU were calculated by multiplying the total volume by the CFU. The mean and standard deviation of data from the triplicate samples was calculated and recorded using an IBM PC equipped with Microsoft Excel, version 5.0.

Toxin Production: Toxin production was estimated in the following manner: The means of the triplicate ELISA results were calculated. Linear regression analysis of the sample means was performed using the data from the standard curve on a Casio X-4000P scientific calculator. The results were recorded and the mean and standard deviation was calculated from the data. Total toxin produced was estimated by multiplying the toxin per ml times the total volume. Toxin per gram of sample was calculated by dividing the total toxin by the grams of sample. Mean and standard deviations were calculated for both total toxin and toxin per gram sample using an IBM PC equipped with Microsoft Excel, version 5.0.

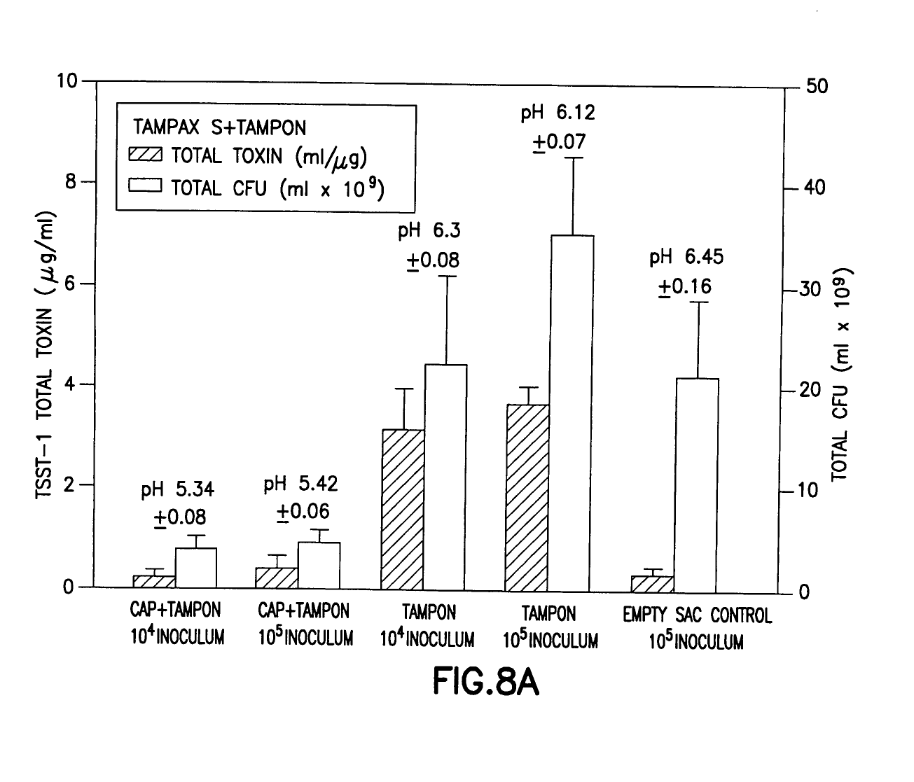

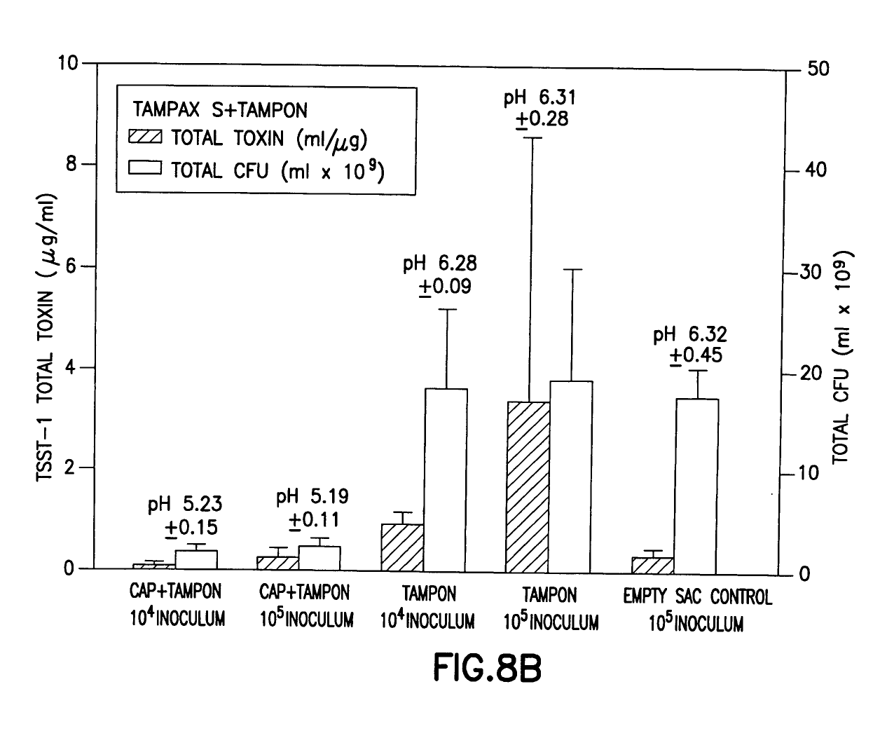

Results shown in FIG. 8 indicate that incorporation of CAP into the tampons greatly suppressed the production of S. aureus and of TSST-1. The incorporation of the CAP film into the tampons also markedly increased their absorptive capacity (by about 70%) and caused a decrease of the pH of the environment by about 1 pH unit (see FIG. 8). In conclusion, incorporation of CAP film into tampons markedly increases their quality by providing increased absorptive capacity and an environment much less likely to allow the propagation of S. aureus and the formation of TSST-1 as well as of other infections of the female reproductive tract, which are not compatible with an acidic environment.

The instant specification is set forth by way of illustration and not limitation, and that various indications and changes may be made without departing from the spirit and scope of the present invention.

What is claimed is:

1. In a tampon including an absorbent material, wherein the improvement comprises an insoluble micronized cellulose acetate phthalate powder being incorporated on or in the tampon, or a tampon wrapped in a cellulose acetate phthalate film, the cellulose acetate phthalate being in a sufficient amount and positioned on the tampon or other feminine hygiene product to prevent the propagation of Staphylococcus aureus and toxic shock syndrome toxin 1 production during menstruation.

2. A method for preventing the propagation of Staphylococcus aureus and toxic shock syndrome toxin I production during menstruation comprising vaginally administering to a human female in need thereof the tampon according to claim 1.

3. A method for maintaining a low vaginal pH comprising vaginally administering to a human female a pharmaceutically effective amount of (i) an insoluble micronized form of cellulose acetate phthalate in the form of a gel, or (ii) a solid dosage form of cellulose acetate phthalate which is dispersible in water, which generates micronized cellulose acetate phthalate particles and is in a form of a film or a sponge.

4. The method according to claim 3, wherein said insoluble micronized form of a cellulose acetate phthalate is administered in the form of a gel.

5. The method according to claim 4, wherein the gel is delivered from a dual compartment applicator in which cellulose acetate phthalate in micronized form is separated from a water-based gel during storage, and the contents of the two compartments are mixed before delivery.

6. The method according to claim 3, wherein said solid dosage form of cellulose acetate phthalate which is dispersible in water is administered in the form of a film or sponge.

7. A method for prevention of shedding and dissemination of a herpesvirus comprising vaginally administering to a human female a pharmaceutically effective amount of (i) an insoluble micronized form of cellulose acetate phthalate in the form of a gel, or a solid dosage form of cellulose acetate phthalate which is dispersible in water, which generates micronized cellulose acetate phthalate particles and is in the form of a film.

8. The method according to claim 7; wherein said insoluble micronized form of cellulosic acetate phthalate is administered in the form of a gel.

9. The method according to claim 8, wherein the gel is delivered from a dual compartment applicator in which cellulose acetate phthalate in micronized form is separated from a water-based gel during storage, and the contents of the two compartments are mixed before delivery.

10. The method according to claim 7, wherein said solid dosage form of cellulose acetate phthalate which is dispersible in water is administered in the form of a film.

11. The method according to claim 7, wherein the herpesvirus is herpesvirus type 2.

12. The method according to claim 8, wherein the herpesvirus is herpesvirus 2.

13. The method according to claim 10, wherein the herpesvirus is herpevirus 2.

14. A method for treating herpes lesions comprising topically administering to a human in need thereof a pharmaceutically effective amount of an insoluble micronized form of cellulose acetate phihalate in the form of a gel.

15. A method for treating herpes lesions comprising topically administering to a human in need thereof a pharmaceutically effective amount of cellulose acetate phthalate film to a premoistened application site.

16. A method for maintaining a normal acidic vaginal environment comprising vaginally administering to a human female a pharmaceutically effective amount of (i) an insoluble micronized form of cellulose acetate phthalate and (ii) Lactobacilli.

17. A method of regularly maintaining a normal acidic vaginal environment and decreasing the replication of harmful microorganisms and virus in the vagina comprising administering to a human female a pharmaceutically effective amount of a micronized cellulose acetate phthalate in the form of a gel, a film or a sponge.

18. A composition comprising micronized cellulose acetate phthalate and 2 to 5 wt % of miconazole nitrate.

19. A method for providing a low vaginal pH, treating herpesvirus, treating a vaginal bacterial infection, blocking toxic shock syndrome generation during menstruation or treating Candida albicans infections comprising administering to a human female a pharmaceutically effective amount of the composition according to claim 18.
